(19) United States
(12) Patent Application Publication
(10) Pub. No.: US 2015/0132189 A1
(43) Pub. Date: May 14, 2015

(54) WIDE BOTTOM DISINFECTING DEVICE

(71) Applicant: Joseph Genaro Alonzo, Tomball, TX (US)

(72) Inventor: Joseph Genaro Alonzo, Tomball, TX (US)

(21) Appl. No.: 14/078,695

(22) Filed: Nov. 13, 2013

(51) Int. Cl. A61L 2/22

(52) U.S. Cl. CPC A61L 2/22
USPC 422/117

(57) ABSTRACT

A wide bottom disinfecting device to disinfect a room comprising: a housing, where the housing includes a wide bottom to ensure that the housing remains upright during use; an actuator on a top portion of the housing; and a disinfecting liquid compressed within the housing, where the disinfecting liquid becomes an aerosolized spray once the actuator is pressed, and where the aerosolized spray is dispersed in the room. The housing may be a cylindrical canister in a wide bottom holder, or a conical canister with a wide bottom portion and a flat top portion.

### BACKGROUND OF THE INVENTION

1. Field of the Invention

The present invention relates to a canister with an extended width base to enable a user to disinfect their home without requiring an expert.

2. Description of Related Art

Many people are concerned with the variety of germs and bacteria that may be present in their homes. The pathogens may cause illnesses like a cold or flu, or may contribute to amplifying allergies already experienced by the occupants. This may be especially concerning to caretakers of children or the elderly. Babies and small children continuously put their hands in their mouth thus transferring whatever germs present on the touched surface into their immature immune systems. Elderly individuals face similar concerns with airborne pathogens because their immune system may be weakened due to preexisting illness, medical treatments and age.

Various products are available to reduce and even eliminate the bacteria such as disinfecting sprays, and cleansing liquids. The cleansing liquids is applied to a surface and then wiped away with a sponge or cloth. The user may spray a toilet, doorknob, countertop and other such surface where bacteria are common. The disinfecting sprays are used by dispersing the spray within the room to kill and sanitize the airborne pathogens. The spray may evaporate killing the pathogen in its wake. Alternatively, the spray may settle and then wiped away with a cloth similar to how the liquids are removed. Typically, the liquids and sprays must be manually applied requiring the user to move through the house to apply the disinfectant to the individual surfaces and rooms. This is often time consuming, tedious and health hazardous to the user because of the requirement for repeated application and close proximity required to use the manually pumped sprays.

It would be beneficial in the art to provide an automatic spray to eliminate germs and bacteria in a room. It would also be beneficial in the art to provide an automatic spray that may be used without continuous monitoring by the user.

### SUMMARY OF THE INVENTION

In view of the foregoing disadvantages inherent in the prior art, the general purpose of the present invention is to provide a wide bottom disinfecting device to disinfect a room, configured to include all of the advantages of the prior art, and to overcome the drawbacks inherent therein.

Accordingly, an object of the present invention is to provide a disinfecting device that automatically disperses a disinfecting spray into a room.

Another object of the present invention is to provide a disinfecting device with a wide base to ensure that the device does not tip or fall over during use.

To achieve the above objects, in an aspect of the present invention, a wide bottom disinfecting device is described comprising: a housing, where the housing includes a wide bottom to ensure that the housing remains upright during use; an actuator on a top portion of the housing; and a disinfecting liquid compressed within the housing, where the disinfecting liquid becomes an aerosolized spray once the actuator is pressed, and where the aerosolized spray is dispersed in the room. The housing may be a cylindrical canister in a wide bottom holder, or a conical canister with a wide bottom portion and a flat top portion.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings in which:

FIG. 1 depicts a perspective view of a disinfecting canister with a holder in accordance with an exemplary embodiment of the present invention; and

FIG. 2 depicts a perspective view of a wide base disinfecting canister in accordance with an alternative embodiment of the present invention.

### DETAILED DESCRIPTION OF THE DRAWINGS

The present invention relates to a canister with an extended width base to enable a user to rid their home of germs and bacteria without requiring an expert. The present invention provides a wide bottom disinfecting device which allows a user quickly fumigate large enclosed spaces. The wide bottom disinfecting device includes a compressed disinfecting liquid which is released upon pressing an automatic trigger. The wide bottom disinfecting device may be embodied as a standard canister with a separate holder, or a wide bottom canister. The canisters are designed so that they do not fall or tip during use. The wide bottom disinfecting device enables user to self-fumigate homes, offices and the like without the service of experts.

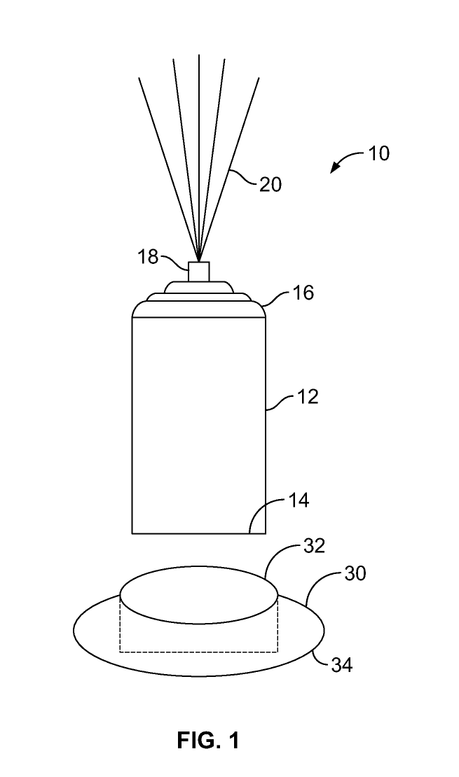

Turning now descriptively to the drawings, referring to FIG. 1, a perspective view of a disinfecting canister 10 with a holder 30 is shown in accordance with an exemplary embodiment of the present invention. The disinfecting canister 10 comprises a cylindrical housing 12 extending between a bottom portion 14 and a top portion 16. The cylindrical housing 12 is made from aluminum to provide a lightweight and transportable structure. Within the cylindrical housing 12 is a compressed disinfecting liquid.

The top portion 16 of the cylindrical housing 12 includes an actuator 18. When pressed the actuator 18 releases the compressed disinfecting liquid as an aerosolized disinfecting spray 20. The aerosolized spray 20 is dispersed like a fountain where it shoots straight up and falls as a 360° dome around the cylindrical housing 12. The actuator 18 may lock into the top portion 16 after being pressed to automatically disperse the aerosolized spray 20 into a room without continuous supervision by a user.

The disinfecting canister 10 includes a holder 30 to ensure that the cylindrical housing 12 remains upright during use. At a top of the holder 30 is an opening 32 to receive the bottom portion 14 of the cylindrical housing 12. The holder 30 includes a wide bottom 34. The wide bottom 34 ensures that the disinfecting canister 10 does not tip or fall when resting within the opening 32. The holder 30 may be made from plastic or aluminum so that it is cheap to make and easily transported with the disinfecting canister 10. The bottom portion 14 may be 2 inches in width where the opening 32 of the holder 30 is a complementary width. The wide bottom 34 of the holder 30 may be 3 inches in width.

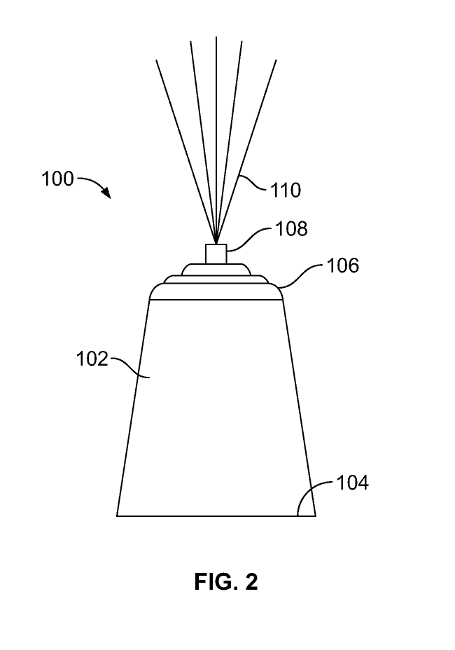

Referring now to FIG. 2 a perspective view of a wide base disinfecting canister 100 is shown in accordance with an alternative embodiment of the present invention. The wide base disinfecting canister 100 comprises a conical housing 102 which includes a wide bottom portion 104 and a flat top portion 106. The conical canister 102 is filled with the same compressed liquid as the cylindrical canister 12. The flat top portion 106 includes an actuator 108 to disperse an aerosolized disinfecting spray 110 in a dome shaped fashion around the conical canister 102. The actuator 108 may lock into the flat top portion 106 to automatically disperse the spray 110 without a user present.

The wide bottom portion 104 of the conical housing 102 enables the wide base disinfecting canister 100 to remain in an upright position during use. Instead of a separate structure, like the holder 30 and the cylindrical housing 12, the conical canister 102 includes a wider bottom 104 than its flat top 106. The wide bottom portion 104 may be 3 inches in width while the flat top portion may be 2 inches.

During use the user may select between the cylindrical canister 10 or the wide base/conical canister 100. The canister is placed in a central portion of the room the user wants to disinfect. If using the cylindrical canister 10 the user may place the holder 30 first and then position the bottom portion 14 of the cylindrical housing 12 within the opening 32 to ensure the canister 10 remains upright. The user then presses the actuator and locks it into the top portion. The user is able to leave the room because the actuator is automatic to continuously disperse the disinfecting spray. Once the canister is emptied of all the disinfecting spray the canister may be discarded. By utilizing the canisters the user is afforded a disinfecting device to eliminate harmful pathogens throughout the room. The canisters do not tip during use to ensure that the disinfecting spray is well dispersed onto all surfaces.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A wide bottom disinfecting device to disinfect a room comprising:
a. a housing, where the housing includes a wide bottom to ensure that the housing remains upright during use;
b. an actuator on a top portion of the housing; and
c. a disinfecting liquid compressed within the housing, where the disinfecting liquid becomes an aerosolized spray once the actuator is pressed, and where the aerosolized spray is dispersed in the room.

2. The wide bottom disinfecting device according to claim 1, where the actuator locks into the top portion of the housing to automatically disperse the aerosolized spray.

3. The wide bottom disinfecting device according to claim 1, where the aerosolized spray is dispersed in a domed shape around the housing.

4. The wide bottom disinfecting device according to claim 1, where the housing is cylindrical canister, and where the wide bottom is a wide bottom holder.

5. The wide bottom disinfecting device according to claim 4, where the wide bottom holder includes an opening to receive a bottom portion of the cylindrical canister.

6. The wide bottom disinfecting device according to claim 1, where the housing is conical canister with a flat top portion and a wide bottom portion.

7. A method of use for a wide bottom disinfecting device to disinfect a room comprising the steps of:
a. placing a housing of the wide bottom disinfecting device in a middle of the room;
b. pressing an actuator on the housing to disperse a compressed disinfecting liquid within the housing as a disinfecting spray;
c. locking the actuator within a top portion of the housing to continue the dispersion of disinfecting spray; and
d. evacuating the room until the wide bottom disinfecting device is empty.

8. The method of use for the wide bottom disinfecting device according to claim 7 further including the steps of:
a. placing a wide bottom holder in the middle of the room; and
b. positioning a cylindrical canister within an opening in the wide bottom holder.

9. The method of use for a wide bottom disinfecting device according to claim 7, where the housing is a conical canister, where the conical canister includes a wide bottom portion and a flat top portion.
